(19) United States
(12) Patent Application Publication
(10) Pub. No.: US 2015/0011648 A1
(43) Pub. Date: Jan. 8, 2015

(54) PROCESS FOR THE MAINTAINING OF A RATIO OF ISOMERS OF CAROTENOID COMPOUNDS

(71) Applicants: Dan Pertig, Halle (Saale) (DE); Christian Schäfer, Basel (CH); Joachim Ulrich, Halle-Wittenberg (DE); Elodie Verdurand, Sisseln (CH)

(72) Inventors: Dan Pertig, Halle (Saale) (DE); Christian Schäfer, Basel (CH); Joachim Ulrich, Halle-Wittenberg (DE); Elodie Verdurand, Sisseln (CH)

(73) Assignee: DSM IP ASSETS B.V., TE Herleen (NL)

(21) Appl. No.: 14/232,988

(22) PCT Filed: Jul. 6, 2012
(86) PCT No.: PCT/EP2012/063262

§ 371 Date: Sep. 5, 2014

(30) Foreign Application Priority Data
Jul. 15, 2011 (EP) 11174194.8

(51) Int. Cl. A61K 31/122; A23K 1/16; A23L 1/30

(52) U.S. Cl. CPC A61K 31/122; A23L 1/30; A23K 1/1609; A23V 2002/00
USPC 514/691

(57) ABSTRACT

The present invention relates to process for the maintaining of a ratio stereoisomers of carotenoid compounds, to specific formulations and to the use of such specific formulations.

The present invention relates to a process for the maintaining of a ratio of stereoisomers of carotenoid compounds, to specific formulations and to the use of such specific formulations.

Carotenoids (which are also named carotenoid compounds in the context of the present patent application) generally consist of conjoined units of the hydrocarbon isoprene, with alternating single and double bonds. The carotenoids absorb light energy of certain frequencies and transfer it to chlorophyll for use in photosynthesis.

Carotenoids are nutritionally important for animals as well as human beings, and also have antioxidant properties.

The carotenoids are classified into two groups:
carotenes (do only contain H and C atoms), and
xanthophylls (do also contain O-atoms).

The carotenoids, when found in nature, are mainly existing in the all-trans stereoisomeric form. But the carotenoids have a tendency to steroisomerise (that means to transform into other stereoisomeric forms). In the context of the present invention E-forms, Z-forms and mixtures of E/Z-forms are meant by stereoisomeric forms. Stereoisomerisation usually results in a mixture of many different stereoisomeric structures: such as:

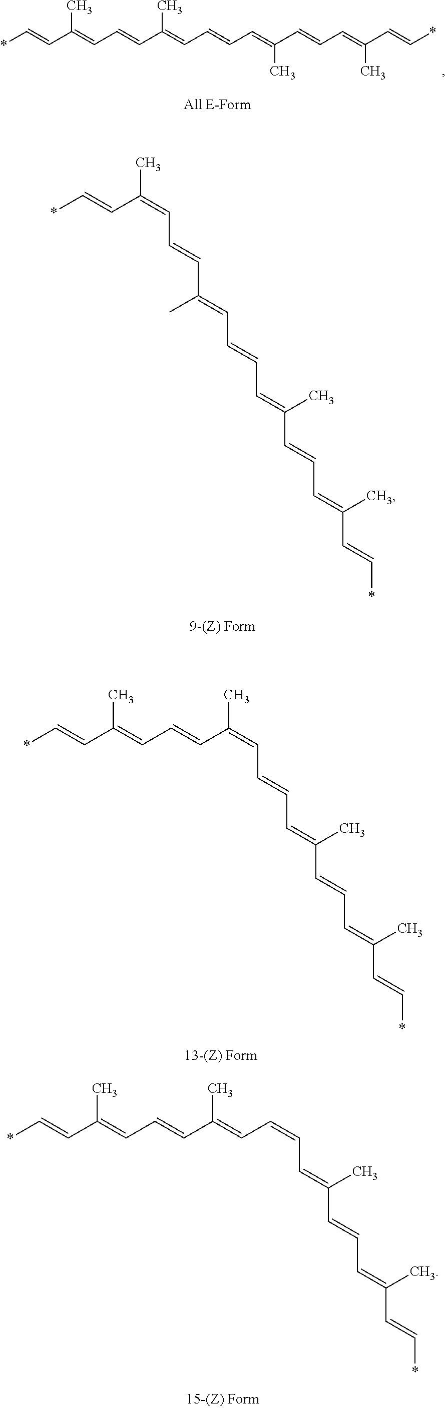 [SMILES: */C=C/C(C)=C/C=C/C(C)=C/C=C/C=C(C)/C=C/C=C(C)/C=C/* ; "," ; */C=C/C(C)=C\C=C\C(C)=C\C=C\C=C(C)\C=C\C=C(C)\C=C\* ; */C=C/C(C)=C/C=C/C(C)=C\C=C\C=C(C)\C=C\C=C(C)\C=C\* ; */C=C/C(C)=C/C=C/C(C)=C/C=C\C=C(C)\C=C\C=C(C)\C=C\*]

The “*”'s (=asterisks) are the positions of the endgroups (R₁ and R₂)

All stereoisomeric forms of the carotenoid compounds usually have different properties in regard to i.e. light absorption, bioavailability etc. So when the isomerisation takes place the properties of a formulation comprising carotenoid compounds can change.

Therefore the goal of the present invention was to find a process, which maintains a ratio of stereoisomers of carotenoid compounds, so that the properties of a formulation comprising carotenoids are not changing.

Surprisingly, it was found that the isomerisation of carotenoid compounds is influenced by the addition of at least one alkali and/or earth alkali metal salt. This addition results that the stereoisomeric form (or also a mixture of two or more stereoisomeric forms) are stabilised. The stereoisomerisation is prevented or slowed down.

Therefore, the present invention relates to a process for the maintaining of a ratio of stereoisomers of carotenoid compounds of formula (I)

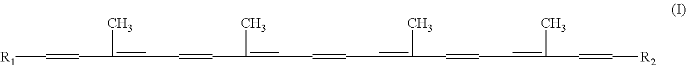

wherein R₁ and R₂ are independently of each other

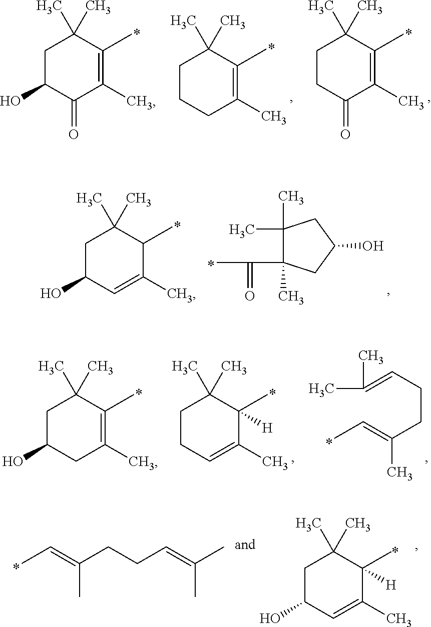

characterised in that at least one compound of formula (I)
is mixed with less than 1 weight-% (wt-%), based on the total weight of the compound(s) of formula (I), of at least one alkali and/or earth alkali metal salt.

The asterisks mark the bond to the backbone. Usually the carotenoid compounds are present in the form of a solution or emulsion. The solution or emulsion can then in a further step be dried (spray dried, freeze dried), mixed with other formulations or ingredients, etc.

The concentration of the carotenoid compound in such a formulation can vary depending on the use of such a formulation. Suitable solvents are such wherein carotenoid compounds are soluble, such as ethylacetate, methylenechloride, chloroform, acetone, etc.

If an emulsion system is used, then it is usually an oil-in-water emulsion. For such an emulsion any commonly known and used oils and emulsifiers are used. The choice of the oil as well as the emulsifier depends on the use of the formulation. In case a food or feed product is produced, then these compounds need to be food or feed grade.

Suitable oils can be from any origin. They can be natural, modified or synthetic. If the oils are natural, they can be plant or animal oils. Suitable oils are i.e. coconut oil, corn oil, cottonseed oil, olive oil, palm oil, peanut oil, canola oil, safflower oil, sesame oil, soybean oil, sunflower oil, hazelnut oil, almond oil, cashew oil, macadamia oil, mongongo nut oil, pracaxi oil, pecan oil, pine nut oil, pistachio oil, sacha Inchi (Plukenetia volubilis) oil, walnut oil, polyunsaturated fatty acids (such as triglyceride and/or ethyl ester, (for example arachidonic acid, eicosapentaenoic acid, docosahexaenoic acid and γ-linolenic acid and/or ethyl ester).

Any commonly known and used emulsifier can be used. The emulsifier can be chosen depending on the final use of the formulation afterwards. Suitable emulsifiers are i.e. modified (food) starches, pectin, alginate, carrageenan, furcellaran, chitosan, maltodextrin, dextrin derivatives, celluloses and cellulose derivatives (e.g. cellulose acetate, methyl cellulose, hydroxypropyl methyl cellulose), lignosulfonate, polysaccharide gums (such as gum acacia, gum arabic, flaxseed gum, ghatti gum, tamarind gum and arabinogalactan), gelatine (bovine, fish, pork, poultry), plant proteins (such as concentrates, isolates, hydrolysates, etc. from peas, soybeans, castor beans, cottonseed, potatoes, sweet potatoes, manioc, canola, sunflowers, sesame, linseed, safflower, lentils, nuts, wheat, rice, maize, barley, rye, oats, lupin and sorghum), animal proteins including milk or whey proteins, lecithin, polyglycerol ester of fatty acids, monoglycerides of fatty acids, diglycerides of fatty acids, sorbitan ester, PG ester and sugar ester (as well as derivatives thereof).

The starches can be modified physically and chemically. Pregelatinized starches are examples of physically modified starches.

Acidic modified, oxidized, OSA-modified, cross-linked, starch esters, starch ethers and cationic starches are examples of chemically modified starches.

The formulations can comprise further auxiliary agents. Depending for which use the formulations are used, the auxiliary agent(s) can vary. These auxiliary agents can be useful for the formulation by further improving its properties, such as physical stability, storage stability, visual perception, etc. Auxiliaries can also be useful for the application in the food, feed or personal care product by improving the property of these compositions, physical stability, storage stability, visual perception, controlled release in the GI-tract, pH control, oxidation resistant, etc. The concentration of these auxiliaries can vary, depending on the use of these auxiliaries. Preferred carotenoid compounds are the following ones:

### Compound of Formula (Ia):

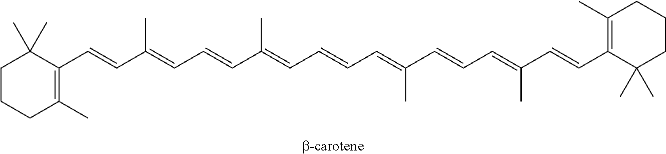 [SMILES: CC1=C(/C=C/C(C)=C/C=C/C(C)=C/C=C/C=C(C)/C=C/C=C(C)/C=C/C2=C(C)CCCC2(C)C)C(C)(C)CCC1]

### Compound of Formula (Ib):

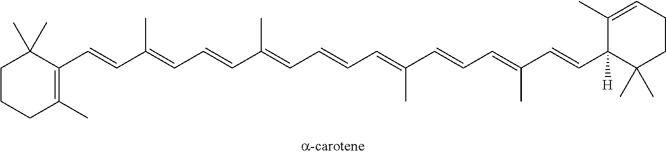 [SMILES: CC1=CCCC(C)(C)[C@H]1/C=C/C(C)=C/C=C/C(C)=C/C=C/C=C(C)/C=C/C=C(C)/C=C/C1=C(C)CCCC1(C)C]

### Compound of Formula (Ic):

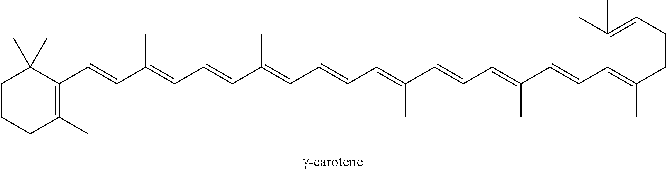 [SMILES: CC(C)=CCC/C(C)=C/C=C/C(C)=C/C=C/C(C)=C/C=C/C=C(C)/C=C/C=C(C)/C=C/C1=C(C)CCCC1(C)C]

### Compound of Formula (Id):

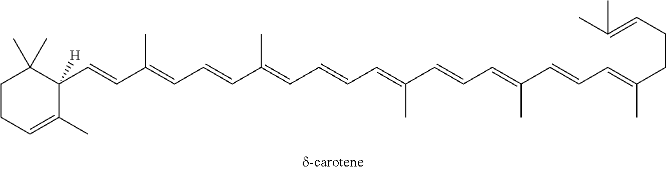 [SMILES: CC(C)=CCC/C(C)=C/C=C/C(C)=C/C=C/C(C)=C/C=C/C=C(C)/C=C/C=C(C)/C=C/[C@H]1C(C)=CCCC1(C)C]

### Compound of Formula (Ie):

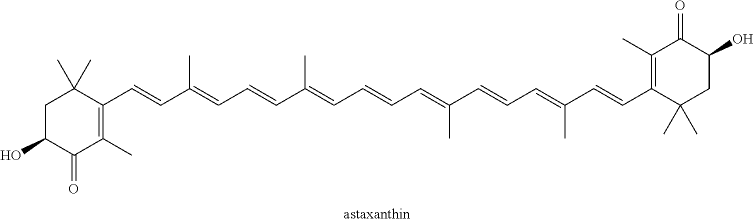 [SMILES: CC1=C(/C=C/C(C)=C/C=C/C(C)=C/C=C/C=C(C)/C=C/C=C(C)/C=C/C2=C(C)C(=O)[C@@H](O)CC2(C)C)C(C)(C)C[C@H](O)C1=O]

### Compound of Formula (If):

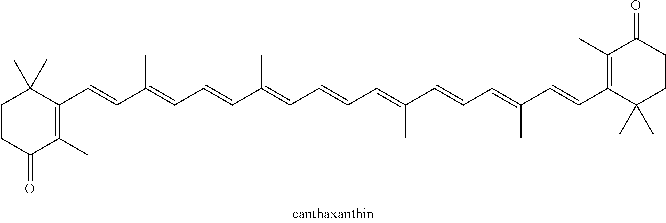 [SMILES: CC1=C(/C=C/C(C)=C/C=C/C(C)=C/C=C/C=C(C)/C=C/C=C(C)/C=C/C2=C(C)C(=O)CCC2(C)C)C(C)(C)CCC1=O]

### Compound of Formula (Ig):

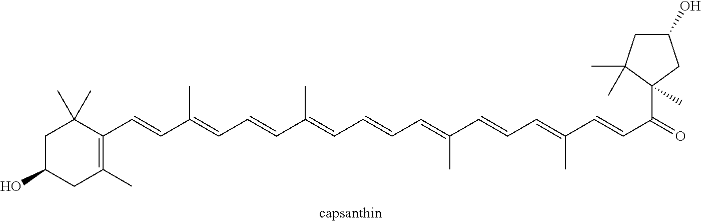 [SMILES: CC1=C(/C=C/C(C)=C/C=C/C(C)=C/C=C/C=C(C)/C=C/C=C(C)/C=C/C(=O)[C@]2(C)C[C@@H](O)CC2(C)C)C(C)(C)C[C@H](O)C1]

### Compound of Formula (Ih):

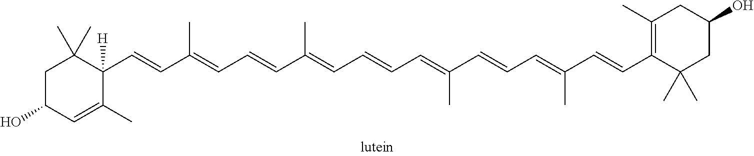 [SMILES: CC1=C[C@H](O)CC(C)(C)[C@H]1/C=C/C(C)=C/C=C/C(C)=C/C=C/C=C(C)/C=C/C=C(C)/C=C/C1=C(C)C[C@@H](O)CC1(C)C]

### Compound of Formula (Ii):

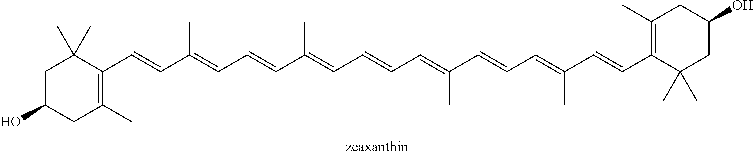 [SMILES: CC1=C(/C=C/C(C)=C/C=C/C(C)=C/C=C/C=C(C)/C=C/C=C(C)/C=C/C2=C(C)C[C@@H](O)CC2(C)C)C(C)(C)C[C@H](O)C1]

### Compound of Formula (Ij):

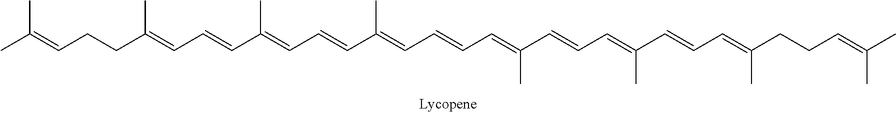 [SMILES: CC(C)=CCC/C(C)=C/C=C/C(C)=C/C=C/C(C)=C/C=C/C=C(C)/C=C/C=C(C)/C=C/C=C(\C)CCC=C(C)C]

To stabilise a ratio of the carotenoid compounds at least one alkali and/or earth alkali metal salt is added in an amount of less than 1 wt-%, based on the total weight of the carotenoid compound or mixture thereof. Preferably less than 0.9 wt-%, more preferably less than 0.8 wt-%. Usually an amount of 0.0001 wt-% to 1 wt-')/0, based on the total weight of the carotenoid compound or mixture thereof, of at least one alkali and/or earth alkali metal is used.

The process is usually carried out at a temperature from 15° C. to 160° C., preferably 20° C. to 130° C., more preferably 20° C. to 110° C.

The process is usually carried out at a pressure of 1 bar to 10 bar, preferably 1 bar to 8 bar, more preferably 1 bar to 5 bar.

Afterwards the carotenoid compounds comprising at least one alkali and/or earth alkali metal salt in an amount of less than 1 wt-%, can be used as such or be used to be further formulated.

The ratio of the isomers is stabilised in the composition as such (only carotenoid compound and alkali and/or earth alkali metal salts) as well in formulations comprising such a composition.

Preferred anions of the alkali and/or earth alkali metal salts are SO₄ or CO₃. Preferred alkali and earth alkali metals of the alkali and/or earth alkali metal salts are K, Ca and Mg.

The most preferred salt is KCO₃.

In case of β-carotene (compound (Ia)), the stereoisomerisation leads to a shift of the shade of the colour. The All Z-form is more reddish whereas the Z forms are slightly yellowish. Therefore a preferred embodiment of the present invention relates to a process for maintaining a ratio of stereoisomers of compound of formula (Ia)

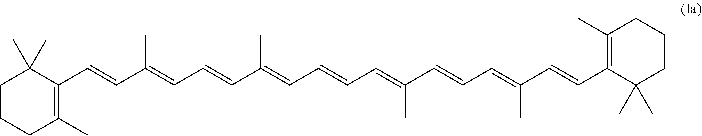 [SMILES: CC1=C(/C=C/C(C)=C/C=C/C(C)=C/C=C/C=C(C)/C=C/C=C(C)/C=C/C2=C(C)CCCC2(C)C)C(C)(C)CCC1]

characterised in that less than 1 wt-%, based on the total weight of the compound of formula (Ia), of KCO₃ is added.

In case of astaxanthin(compound (Ie)), the stereoisomerisation leads to different bioavailability of the compound. The all (E) form of astaxanthin has the best bioavailability. Therefore this form (or a form with a high amount of all (E) at least 85% of all (E)) should be stabilised.

Therefore a preferred embodiment of the present invention relates to a process for maintaining a ratio of stereoisomers of compound of formula (Ie)

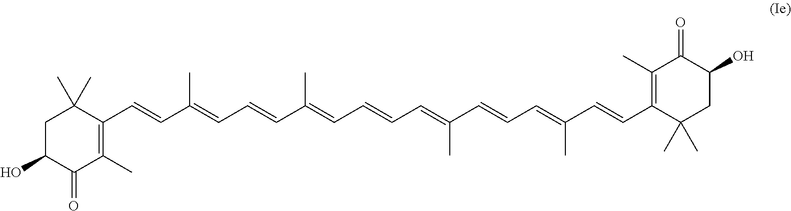 [SMILES: CC1=C(/C=C/C(C)=C/C=C/C(C)=C/C=C/C=C(C)/C=C/C=C(C)/C=C/C2=C(C)C(=O)[C@@H](O)CC2(C)C)C(C)(C)C[C@H](O)C1=O]

characterised in that less than 1 wt-%, based on the total weight of the compound of formula (Ie), of KCO₃ is added.

The alkali and/or earth alkali salt can be added as a solid form as well as a solution (or a combination of both).

Furthermore the present invention also relates to a formulation comprising at least one carotenoid compound of formula (I)

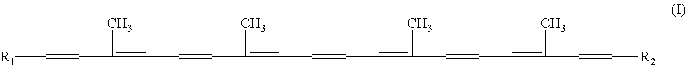

wherein R₁ and R₂ are independently of each other

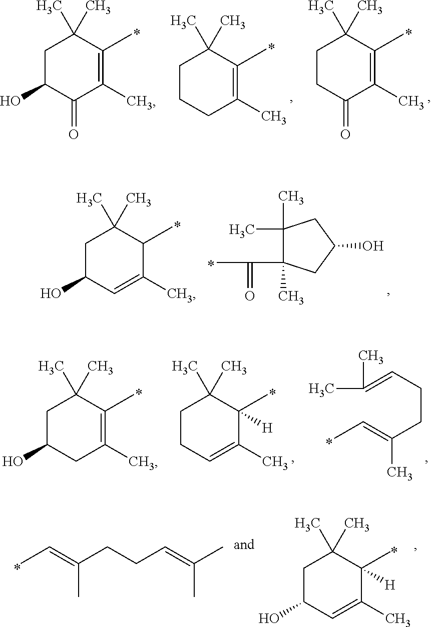

and 0.0001 to 1 wt-%, based on the total weight of the compound(s) of formula (I), of at least one alkali and/or earth alkali metal salt.

All preferences for the process also apply for the formulations.

As said above the formulations the carotenoid compounds comprising at least one alkali and/or earth alkali metal salt in an amount of less than 1 wt-%, can be used as such or used to be further formulated.

The formulation as described above can be used in food, feed or personal care products.

The amount of a formulation as described above used in food, feed or personal care products depends on the food, feed or personal care products

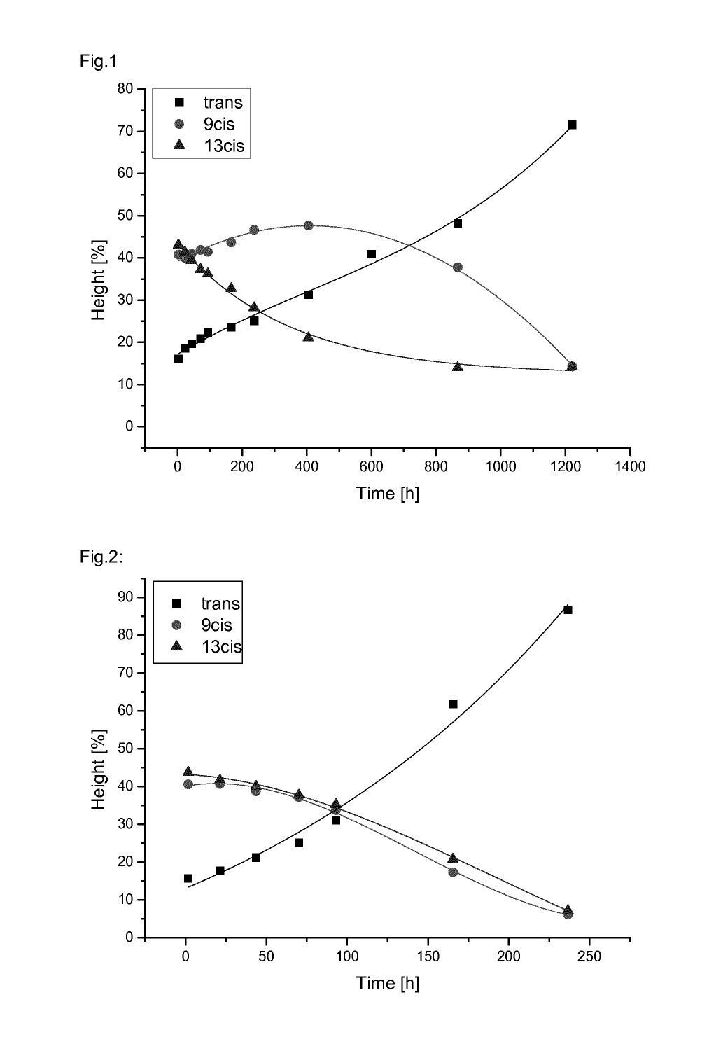

FIG. 1.: Isomerisation of the inventive formulation over a period of more than 50 days (at room temperature)

FIG. 2.: Isomerisation of the comparative formulation over a period of more than 10 days (at room temperature)

The following examples serve to illustrate the invention. All percentages are given in relation to weight and the temperature is given in degree Celsius.

### EXAMPLE 1

200 mg astaxanthin was dissolved in 15 ml ethylacetate. 14 mg KCO₃ was added to the solution. The solution was homogenized and the formulation was tempered at 30° C. The solution was stored and samples were taken periodically and the concentration of astaxanthin was measured spectrophotometrically at 478 nm. FIG. 1 shows the isomerisation of astaxanthin during this period.

### EXAMPLE 2

### Comparative Example

200 mg astaxanthin was dissolved in 15 ml ethylacetate. NO KCO₃ was added. The solution was homogenized and the formulation was tempered at 30° C. The solution was stored and samples were taken periodically and the concentration of astaxanthin was measured spectrophotometrically at 478 nm.

FIG. 2 shows the isomerisation of astaxanthin during this period. It can be seen that the formulation according to example 1 is far better stabilised than the one of example 2.

### EXAMPLE 3

### Preparation of the Water Phase (Formulation (1))

80 g of deionised water are mixed with 135 g of Calcium lignosulfonate and 33.8 g of Yellow dextrin under slow stirring. Afterwards the reaction mixture is stirred at room temperature by using an Ekato® FD stirrer. When the solution is homogenous 10 g of deionised water are added. Then the solution is heated (water/oil bath temperature of 62° C.) and stirred (1200 rpm). Afterwards 20 g of deionised water is added.

Now the pH is adjusted to 7 by adding NaOH (20%). Afterwards the reaction mixture is cooled down (water/oil bath temperature of 30° C.).

### Preparation of the Oil Phase (Formulation (2))

6.8 g of dl-α-tocopherol, 13.5 g of astaxanthin and 0.096 g KCO₃ are mixed under an oxygen free atmosphere in a glovebox. Afterwards 150 ml of dichloromethane are added. The reaction mixture is stirred (500 rpm) and heated up until the astaxanthin and the tocopherol are dissolved completely.

1. Emulsion Step:

Formulation (2) is added to the formulation (1) under vigorous stirring. Afterwards this reaction mixture is stirred under constant heating (water/oil bath temperature of 30° C.) for about 30 minutes. The excess of solvent is removed under reflux. An emulsion (Emulsion I) is obtained.

2. Emulsion Step:

Formulation (2) is added to Emulsion I under stirring and heating (water/oil bath temperature of 30° C.). Afterwards this reaction mixture is stirred under constant heating (water/oil bath temperature of 30° C.) for about 30 minutes. The excess of solvent is removed under reflux. An emulsion (Emulsion II) is obtained.

3. Emulsion Step:

Formulation (2) is added to Emulsion II under stirring and heating (water/oil bath temperature of 30° C.). Afterwards this reaction mixture is stirred under constant heating (water/oil bath temperature of 30° C.) for about 30 minutes. The excess of solvent is removed under reflux. An emulsion (Emulsion III) is obtained. The average particle size is 110 nm.

Beadlet Production

Emulsion III is heated for about 3 hours by using a rotary evaporator. A vacuum of about ca.200 mbar is created. Afterwards the reaction solution is diluted with about 30 ml of water so that the solution has a torque value of about 1.0 Ncm. Afterwards the reaction solution is undergoing a powdercatch process (maize starch as powder catch media).

1. A process for the maintaining of a ratio of stereoisomers of carotenoid compounds of formula (I) 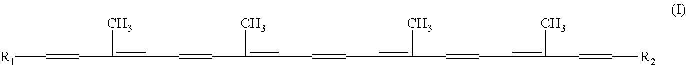
wherein R₁ and R₂ are independently of each other 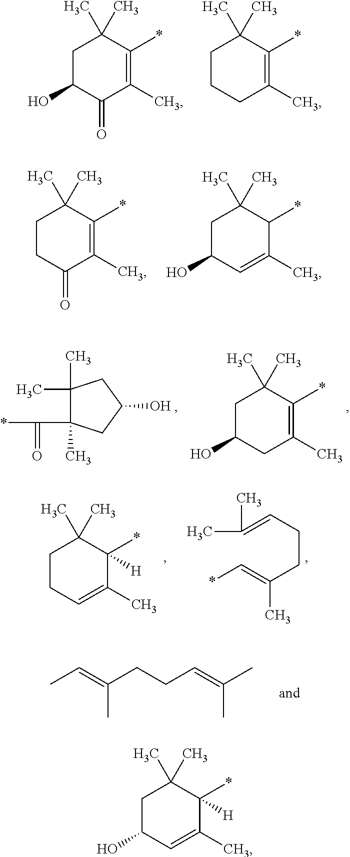
characterised in that the compounds of formula (I)
are mixed with less that 1 wt-%, based on the total weight of the compound(s) of formula (I), of at least one alkali and/or earth alkali metal salt.

2. Process according to claim 1 wherein the carotenoid compounds are chosen from

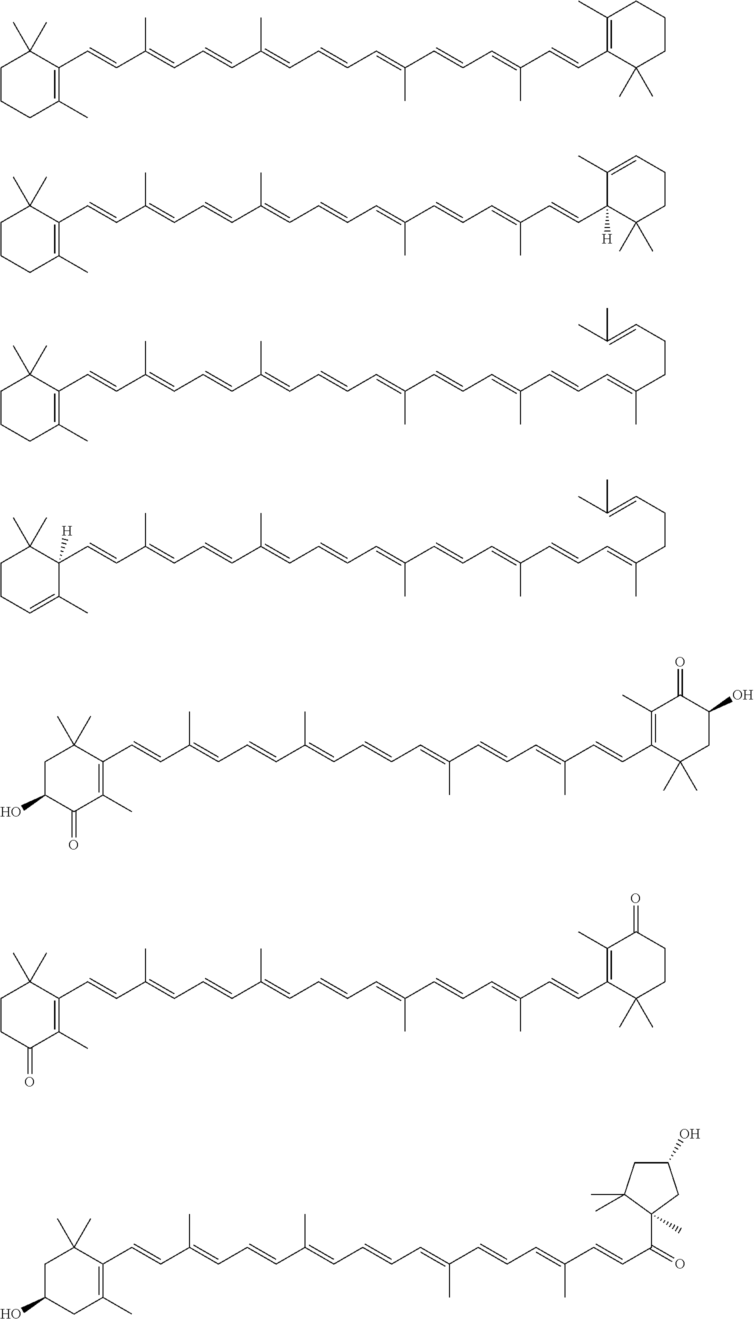

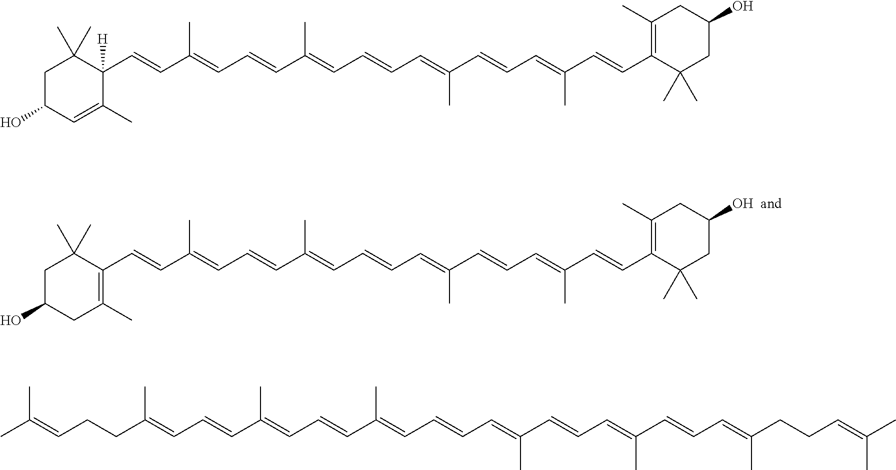

3. Process according to claim 1, wherein the carotenoid compound is

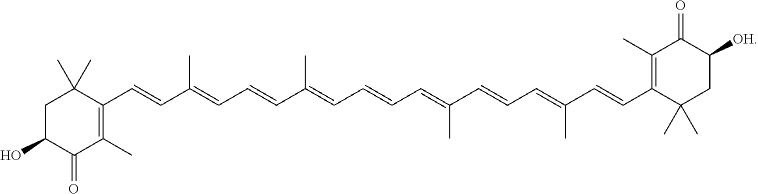 [SMILES: CC1=C(/C=C/C(C)=C/C=C/C(C)=C/C=C/C=C(C)/C=C/C=C(C)/C=C/C2=C(C)C(=O)[C@@H](O)CC2(C)C)C(C)(C)C[C@H](O)C1=O]

4. Process according to claim 1, wherein the carotenoid compound is

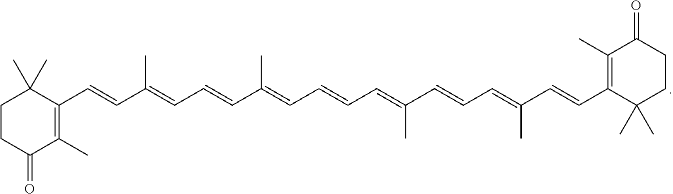 [SMILES: CC1=C(/C=C/C(C)=C/C=C/C(C)=C/C=C/C=C(C)/C=C/C=C(C)/C=C/C2=C(C)C(=O)CCC2(C)C)C(C)(C)CCC1=O]

5. Process according to claim 1 wherein the alkali and/or earth alkali metal salts are SO₄ and/or CO₃ salts.

6. Process according to claim 1, wherein the alkali and/or earth alkali salts are K, Ca and/or Mg salts.

7. Process according to claim 1, wherein KCO₃ used.

8. Process according to claim 1 wherein 0.0001 wt-% to 1 wt. %, based on the total weight of the compound(s) of formula (I), of the alkali and/or earth alkali salt is used.

9. A formulation comprising at least one carotenoid compound of formula (I) 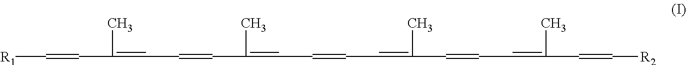
wherein R₁ and R₂ are independently of each other 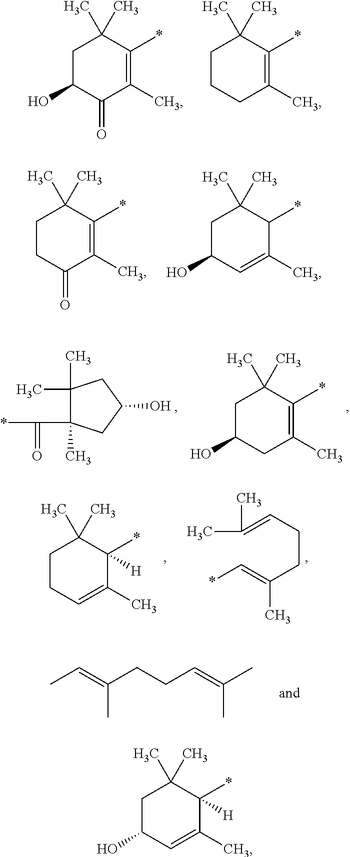
and 0.0001 to 1 wt-%, based on the total weight of the compound(s) of formula (I), of at least one alkali and/or earth alkali metal salt.

10. Formulation according to claim 9 wherein the carotenoid compounds are chosen from

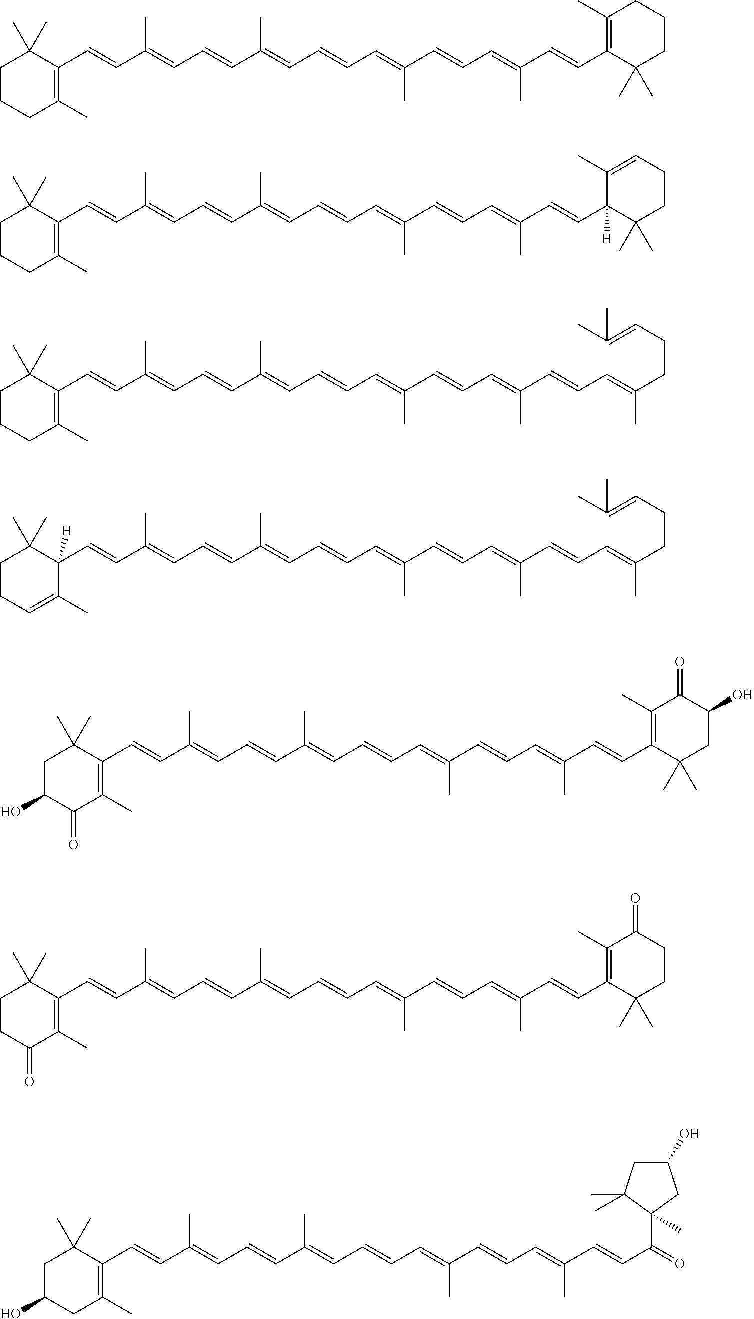

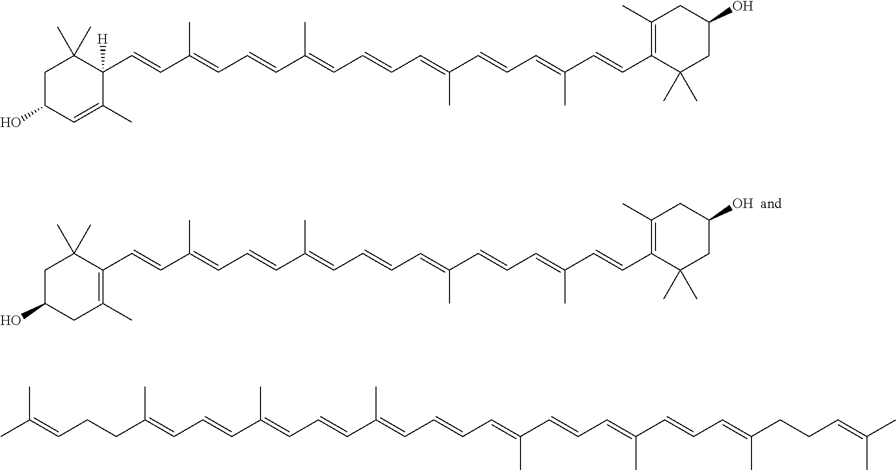

11. Formulation according to claim 9, wherein the carotenoid compound is

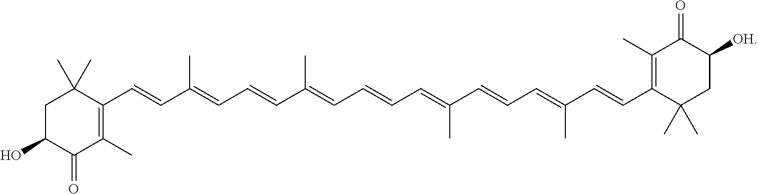 [SMILES: CC1=C(/C=C/C(C)=C/C=C/C(C)=C/C=C/C=C(C)/C=C/C=C(C)/C=C/C2=C(C)C(=O)[C@@H](O)CC2(C)C)C(C)(C)C[C@H](O)C1=O]

12. Formulation according to claim 9, wherein the carotenoid compound is

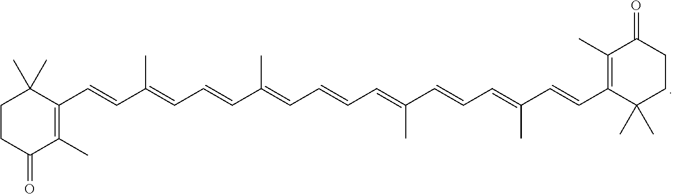 [SMILES: CC1=C(/C=C/C(C)=C/C=C/C(C)=C/C=C/C=C(C)/C=C/C=C(C)/C=C/C2=C(C)C(=O)CCC2(C)C)C(C)(C)CCC1=O]

13. Formulation according to claim 9 wherein the alkali and/or earth alkali metal salts are SO₄ and/or CO₃ salts.

14. Formulation according to claim 9, wherein the alkali and/or earth alkali salts are K, Ca and/or Mg salts.

15. Formulation according to claim 9, wherein KCO₃ is the salt.

16. Formulation according to claim 1 wherein 0.0001 wt-% to 1 wt-%, based on the total weight of the compound(s) of formula (I), of the alkali and/or earth alkali salt is comprised.

17. Use of at least one formulation according to claim 9 in food, feed or personal care products.

18. Food, feed or personal care product comprising at least one formulation of claim 9.
